(19) United States
(12) Patent Application Publication
(10) Pub. No.: US 2002/0045836 A1
(43) Pub. Date: Apr. 18, 2002

(54) Operation of wireless biopotential monitoring system

(76) Inventor: Dima Alkawwas, Urbana, IL (US)

Correspondence Address: Pauley Petersen Kinne & Fejer, Suite 365, 2800 W. Higgins Road, Hoffman Estates, IL, 60195, US

(21) Appl. No.: 09/783,471

(22) Filed: Feb. 14, 2001

Related U.S. Application Data
(63) Continuation of application No. 09/688,442, filed on Oct. 16, 2000, now abandoned.

(51) Int. Cl. A61B 5/0428

(52) U.S. Cl. 600/509

(57) ABSTRACT

Multiple wireless sensor assemblies are individually attached to standard body locations for EKG signal recording. The sensors measure small biopotential signals at short distances on the body sites, and the small signals are used to calculate an output signal that resembles a conventional EKG measurement signal over the long distance between the sensors. An algorithm is employed to calculate the standard EKG signal using the two measurement sites' data and an attenuation value between sensor contacts which has been previously measured.

### CROSS REFERENCE TO RELATED APPLICATIONS

### BACKGROUND OF THE INVENTION

The present application is a continuation of the application, U.S. Ser. No. 09/688,442, filed 16 Oct. 2000.

1. Field of the Invention

This invention relates to the field of devices used to measure and display biopotential signals generated by the body. More specifically, the invention relates to multiple wireless, programmable sensor systems.

2. Discussion of Related Art

Conventional EKG apparatus for biopotential measurements typically require multiple, e.g. up to ten, single point contact conventional electrodes. Each electrode is connected to a wired lead that senses biopotential and transmits it to instrumentation amplifiers which perform a differential voltage potential measurement from the measurement sites. The wires extending from such electrodes present a problem for the patient as well as the health care personnel to manage. Electrode lead wires are time consuming to sort out, provide an opportunity for error of connection to the wrong electrode site, and obstruct the patient mobility, posing a lot of discomfort to wear. Leads also contribute to low signal to noise ratio as they move about.

Telemetry systems in hospitals currently exist, but still require use of wired leads connected to the patient body to obtain measurements. Known telemetry systems amplify and filter such measurements in a unit worn by the patient, which then transmits differential signals to a receiving base station.

Wireless EKG biopotential medical monitoring and diagnosis systems have been described in the prior art. The major problem with such systems lies in the underlying assumptions made on the fundamentals of measurement methodology for EKG biopotential signals. Major assumptions are made about the ability to create localized voltage references at each of the disconnected measurement sites on the body.

Current instrumentation amplifier recording systems for EKGs use a common, i.e. identical, reference point between the two measurement points from which a biopotential is recorded. The common reference point causes this common, or global, reference to cancel out when measuring the differential potential between the two inputs. Thus it does not matter if the potential at the reference is time varying and not fixed since it cancels out exactly by being a common, or identical, reference point to both inputs. The location of the reference on the body for bipolar measurements actually does not affect the measurement since it cancels out, therefore it can be located anywhere on the body. As long as the reference remains common between the two differential inputs, then it is proper to use as a global reference. Having an identical reference point to the two inputs minimizes issues with instabilities or time variation which are of major concern in techniques using independent, separate, localized references.

Known wireless EKG patents Segalowitz 5,307,818 and Besson 5,862,803 are based on either single point contact conventional electrodes, or a dual point contact electrodes. Use of dual point contact electrodes can provide a unipolar biopotential measurement but does not offer as good a common mode rejection ratio (CMRR). In the Segalowitz patent, the reference signal is claimed to be picked up by one of the contact points of the dual contact electrode strip or the outermost ring of the concentric ring electrode. But a problem remains in the time varying alternation (AC variation) between the individual localized references which means that references are ineffective.

In order for unconnected localized references to qualify as a valid reference, they need to be at either an identical potential level or at least be at a DC constant potential from each other. No time varying potential difference relationship should be detected between the local references. The local references also need to maintain a high degree of stability at a specific potential level, with high degree of precision in order to not introduce false components in the measured signal.

The Segalowitz reference also describes one-way communication sensor devices in which the sensor is in either transmit only or receive only mode. The sensors can only be programmed via manual switches on the sensor frame, and not dynamically over-the air from the base station

Neither of the Segalowitz or the Besson patents discuss a required processing algorithm for retrieval of conventional EKG signals from measurements performed with shorter lead distance separated electrodes. Signals measured with suggested techniques in these patents are not believed to yield any standard or conventional EKG signals which is a drawback for integration of the systems into current practice and archival patient histories. Neither the Segalowitz or the Besson patent describe a communication protocol for exchange of information between the sensors and the base station.

There is further a need for improved communication between the wireless EKG sensors of the known art and the receiving, or base station, as well as a need for improved interfacing between the base station and medical personnel, or further data receiving apparatus, or both.

### SUMMARY OF THE INVENTION

The present invention provides a wireless, programmable, EKG biopotential monitoring system for the body from disconnected measurement sites. This allows the removal of all wires that typically have been used to connect the electrical leads to the electrode sites on the body. Multiple, small signal, independent measurements can be taken from completely disconnected sites. The resultant measurements can then be combined mathematically to reproduce conventional lead measurement. The system of the present invention enables miniature, low weight, independent, wireless sensors that are attached to small electrode patches. The sensors can detect small signal EKG measurements, and transmit digital signals to an associated base station where the conventional EKG measurements are reconstructed.

The base station receives the measured EKG biopotential signals, and reconstructs an output signal that resembles conventional EKG measurement signals. The base station synchronizes the multiple sensor measurements, applies signal processing algorithms for reconstruction, calibration, and filtration of the incoming signals, and supplies the signal at the output interface terminal in either digital or analog form. The output EKG signals may be then used as input to either standard EKG monitors, or personal computer systems, where it can be displayed and further analyzed.

It is among the objects of the present invention to provide an EKG, or more generically, a biopotential, monitoring system which can:

1. Interface with existing monitoring equipment and personal computers;

2. Provide greater mobility for the patient by reliably transmitting data over wireless RF connection;

3. Enhance patient comfort by eliminating all wires connecting patient body to monitoring equipment or telemetry boxes;

4. Reduce sensitivity of EKG recording to motion artifacts by eliminating the wires which represent moving components of the EKG system that typically pull or push the electrode pads causing severe signal baseline level variation during motion;

5. Provide cost savings recognized in terms of time invested by health care personnel in connecting, sorting and organizing, and disconnecting patient wire leads;

6. Reduce the number of electrode patches disposed of in the process of continuous monitoring;

7. Allow for extended periods of monitoring with low power wireless sensors in a hospital room environment without having to replace the hospital's existing bedside monitoring equipment;

8. Allow for in-home continuous monitoring of patients; and

9. Store data on the base station for extended period of time and upload data later on to a remote computer or monitoring station providing greater convenience for both the patient and the physician to access and view the patient's history.

The exchange of informational messages between the base station and the wireless sensors is represented by wireless, e.g. RF, signals indicating specific command instructions, or responses, or informational messages, and sensor associated data. The described communication protocol can be used to dynamically program and control the behavior of the wireless sensors. A common data format between the base station and the sensors is designed for a communication protocol. Such communication protocol informational messages may include signals to:

1. Initiate or stop data acquisition;

2. Change signal amplification dynamically to obtain larger signal amplitudes;

3. Change the input range on the A/D converters;

4. Control the resolution of A ID converters;

5. Adaptively adjust power levels for RF transmission;

6. Monitor battery power levels and alarm low battery power;

7. Calibrate performance parameters;

8. Dynamically adjust filtering parameters to reduce noise;

9. Change RF transmission frequency band when experiencing a noisy channel;

10. Increase or decrease the sampling rate used for digitizing measured signals;

11. Synchronize timing for sampling of data measurements;

12. Synchronize data transmission time slots from each of the multiple sensors to the base station;

13. Program unique identification information such as a unique sensor identifier, sensor group identifier, base station identifier, and patient identifier for unique association between the base station, the group of sensors, and the patient;

14. Program patient and physician identifying and contact information in the sensors and base station;

15. Assign electrode labels indicating functional position on the body;

16. Control error recovery process and retransmission of information if necessary;

17. Detect sensor transmission frequency;

18. Run diagnostics to test data acquisition subsystem, data amplification and filtration subsystems, and data transmission subsystem;

19. Manage sensor signal fade away, and paging by the base station until detection and re-establishment of RF connection; and

20. Audit performance and proper operation of various subsystems.

Further aspects of the invention will be discussed in the following detailed description of the preferred embodiment.

### BRIEF DESCRIPTION OF THE DRAWINGS

A preferred embodiment of the invention is described below along with the appended drawing Figures.

FIGS. 1A-1C depict the orientation of electrodes for measurement of a 3-lead EKG and a 4-lead EKG.

FIG. 2 depicts a representation of the 3-lead EKG configuration and the relationship between Lead I, II, and III which form a closed loop electrical circuit.

FIGS. 3A and 3B depict the resistor network model connecting pathways between left arm and right arm, and left arm and left leg, respectively.

FIG. 4 depicts the attenuation measured between A and B, as well as the variation in the measurement between C and D indicated in FIGS. 3A and 3B.

FIG. 5A depicts the propagation of the electric current away from the biological current source, or heart, and outward across the resistive circuit between the connection sites. FIGS. 5B and 5C depict the linearity and parallelism between current vectors illustrated in FIGS. 5D and 5E.

FIGS. 6A, 6B, 6C and 6D depict computed versus measured EKG signals.

FIGS. 7A, 7B, 7C and 7D depict top views of configurations of a three point contact electrode patch according to the present invention.

FIG. 8 depicts multiple layers in the sensor fabrication, the underlying electrodes, electronic assembly, and cover with replaceable battery usable with the three point sensor patch.

FIGS. 9A, 9B and 9C depict the measurement of a single precordial lead in terms of orientation of electrodes and resistive network model.

FIG. 10A depicts a strip of precordial leads with the reference point located about 3 inches away from the V1 position, with FIG. 10B showing the strip on the left side of the body and along the axis of the line connecting to the conventional right leg electrode position.

FIG. 11 depicts a block diagram of the sensor electronic assembly.

FIG. 12 depicts a block diagram of the base station electronic assembly.

FIGS. 13A, 13B, and 13C depict representations of electrode positioning in existing systems on a patient measuring 3-lead, 4-lead, and 12-lead EKG signals, respectively, with wired leads attached to the electrode.

FIG. 14 depicts the instrumentation amplifier circuit used in obtaining measurement signals from each of the three point contact electrodes.

### DETAILED DESCRIPTION OF THE PREFERRED EMBODIMENT

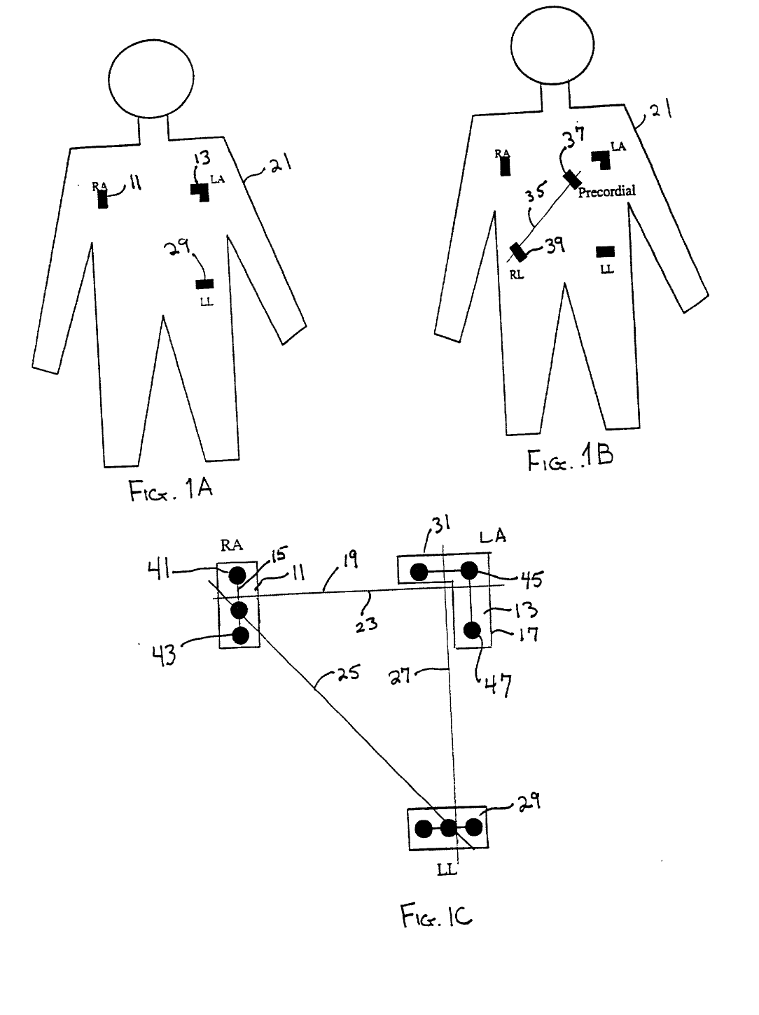

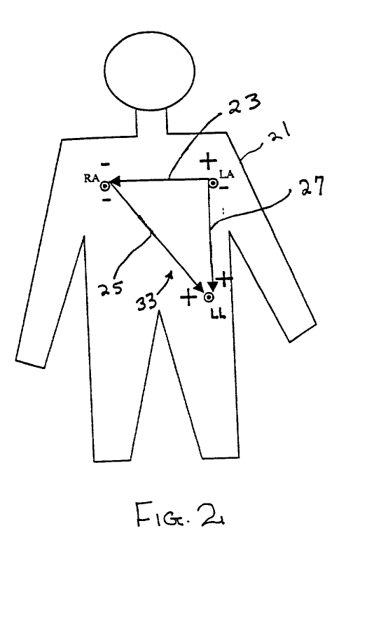

Referencing FIGS. 1A-1C, to measure a biopotential voltage drop between two points of a standard EKG lead, the present invention requires two separate electrode patches, e.g. 11,13 to be placed at the desired sensing points. The electrode patch is aligned with its long axis 15, or arm 17, perpendicular to the axis line 19 connecting the two desired measurement points of the electrodes 11,13. The direction of orientation of the electrode patches is important since biopotential measurements are directionally dependent.

FIG. 1A depicts placement of electrodes on a body 21 for measurement of a 3-lead EKG having Lead I 23, Lead II 25, and Lead III 27. The electrodes 13, 11 and 29 are placed on the left shoulder area ( LA), right shoulder area ( RA), and left hip area (LL) respectively. The second, LA, electrode 13 is a right angle triangle shape with one arm 17 perpendicular to the conventional Lead I axis line 19, and its second arm 31 perpendicular to the conventional Lead III axis line 27. The first and third electrodes 11 and 29 respectively, are collinear line patches with first electrode 11 placed perpendicular to the Lead I axis line 25 and second electrode 29 placed perpendicular to the Lead III axis line 27.

Using the electrodes it is possible to make a computed value of Lead I and Lead III EKG signals measurement, from which is derived another computed estimate of the Lead II EKG signal measurement. The three leads make a closed electric circuit loop 33 in terms of biopotential voltage drop as shown in FIG. 2.

FIG. 1B also depicts the placement of a fourth EKG lead line 35, the precordial lead, with the fourth or precordial electrode 37 and fifth, or right hip (RL) electrode 39 aligned perpendicularly to the axis line connecting the two electrode sites.

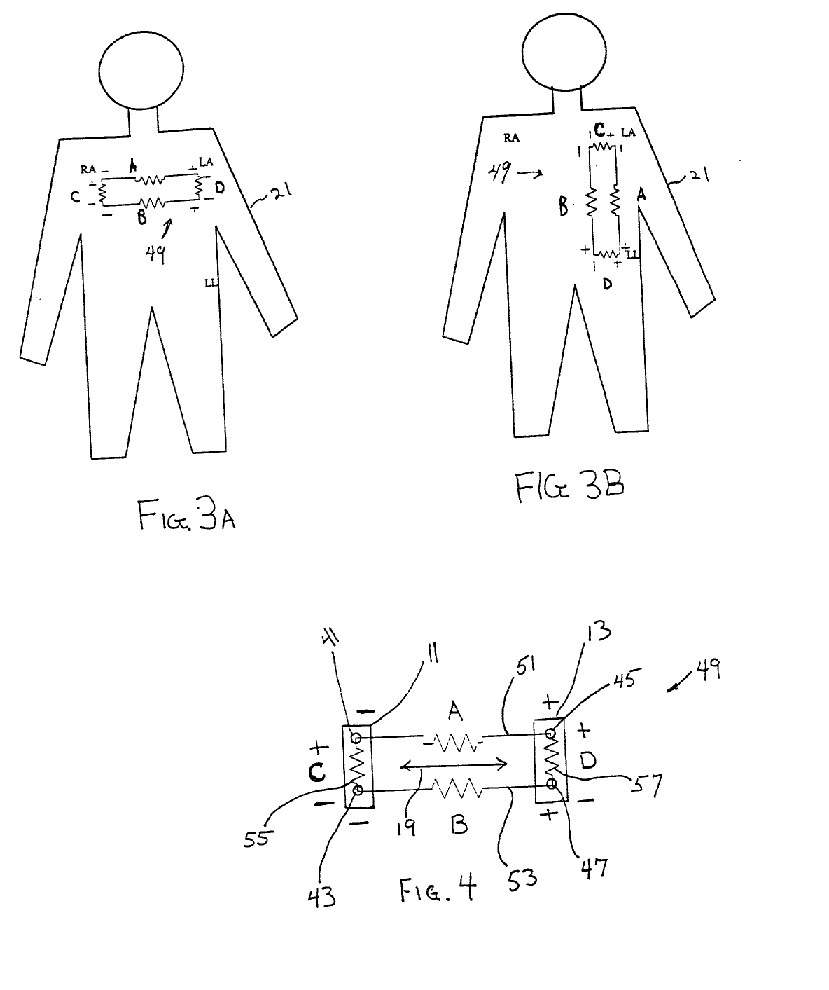

Referencing FIG. 1C, to measure a specific EKG lead potential, three contact points (two inputs and a reference) on two independent electrode patches e.g. 11, 13 are placed on the relevant anatomical locations on the body 21. The two input contacts 41, 43 and 45, 47, respectively, from each electrode 11, 13 are oriented perpendicular to the line of direction of the EKG lead axis-line, e.g. 19. The four input contacts (from both electrodes) thus make up a rectangular circuit diagram 49, as depicted in FIGS. 3 and 4.

Referencing FIG. 4, the comers of the rectangle are represented by the two inputs from each of the two opposing electrode patches. The separation between the two input contact points for each electrode is significantly smaller than the separation distance for the longer sides of the rectangle. For example, for measuring an EKG on an adult human body, on each electrode a contact separation of two inches for adults, and one inch for children is desirable. Different applications may vary this separation distance between 0.5 to 3 inches. Conventional EKG electrode separation distance requires approximately a 12 inch separation for adults.

Thus the problem is to reproduce, or calculate, the biopotential measurement across the longer sides 51, 53 of the rectangle 49 by obtaining only two smaller biopotential measurements across the smaller sides 55,57 of the rectangle 49. The points of contact across the body are still all connected electrically by the patient's skin and body, and the potentials across all of the contact points all add up to zero since they represent a closed electrical circuit loop. The body and skin are modeled as simple resistive conductor elements A, B, C, D representing voltage drops between the contact points 41, 43, 45, 47 from each electrode 11, 13, respectively. Electrode positioning orientation is critical to the measurement obtained, and the rectangular shape 49 provides the measuring electrodes directly perpendicular to the axis of the desired estimated biopotential signal lead line 19.

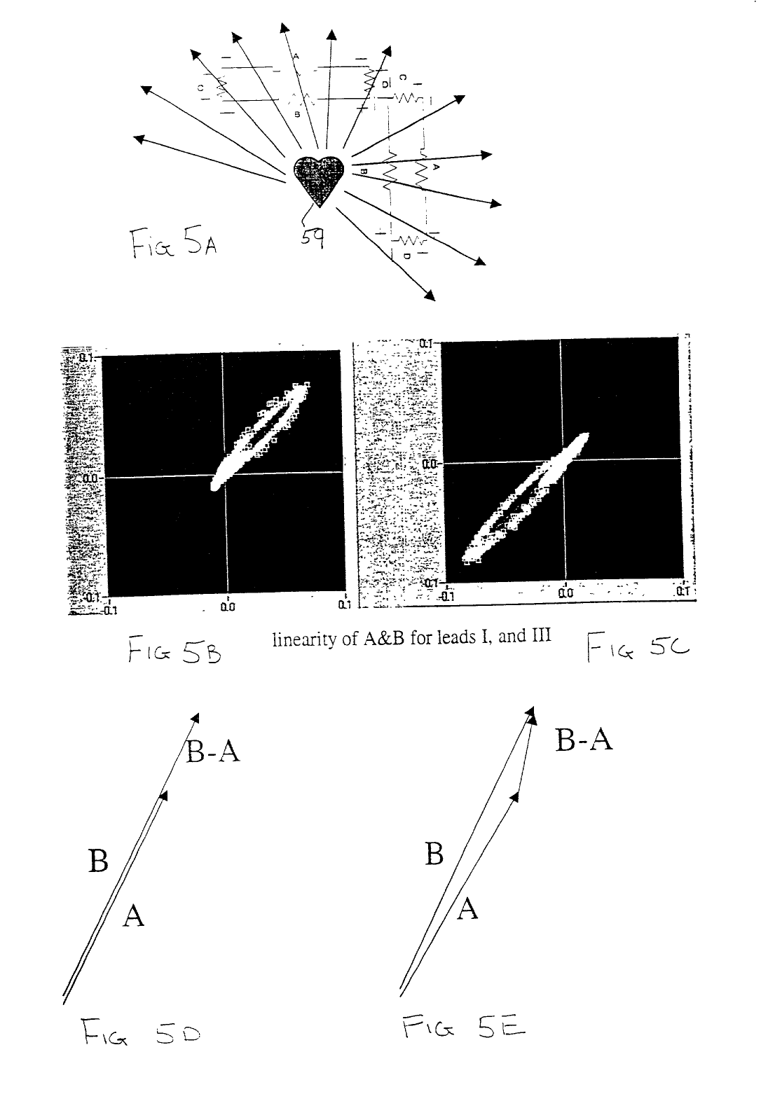

Potential B-A will equal potential C-D. In other words, the difference in measurements across the longer sides 51, 53 of the rectangle equals the difference in measurements across the smaller sides 55, 57 of the rectangle 49. This relationship can also be coupled by another realization that long side biopotential measurement signals lie on either side of the same axis line of the desired EKG lead 19. These signals should therefore be very similar in terms of the frequency content and have a linear relationship with each other. One long side signal will be just an attenuated version of the other and related by a simple first order linear equation of a scaling factor and an offset. The only noticeable difference is a slight increase or decrease in amplitude of the measured EKG signal, due to the attenuation by the body when the signal is measured at a more distant site from the source 59 of the signal, e.g. the heart, as depicted in FIG. 5A.

FIGS. 5B-5E show the effect of this small attenuation factor which results in a decreased amplitude, but the frequency information content (i.e. the time varying nature of the signal) remains the same along the same direction of measurement. Referencing

FIGS. 5D and 5E, representing the measured biopotential signals in vector terms, signals, i.e. time varying voltages, A and B can be described by a norm representing the length of the vector and a unit vector representing the signal itself. Signals A and B have different norm values ∥A∥ and ∥B∥ representing the attenuation relationship between them, but their unit vectors U_{A}, U_{B} are substantially linear and parallel with each other. In other words, the vectors have the same direction but different lengths. That is, signals A and B have a linear relationship with each other that can be described by a simple first order linear equation representing a scaling factor and an offset.

The small separation distance between parallel signal vectors A and B is defined physically by the separation distance between the contacts within the electrodes, as represented by segments C and D, and mathematically by B-A (B being closer to the heart signal source 59). Two considerations must account for selection of the contact distance within the electrodes. The larger the separation distance between the long sides 51, 53 of the rectangle, the less valid becomes the assumption of linearity of the relationship between signals A and B when one is trying to obtain them from calculations. Also, the smaller the separation distance between the long sides 51, 53 of the rectangle, i.e. the smaller the distance between contact points on an electrode, the smaller will be the measurable value of the potential, or voltage, drops C, and D. That is, as skin resistance decreases with smaller lengths, the potential drop may be decreased to nano Volt levels. At such small voltage levels the signal becomes more sensitive to noise and motion artifacts. There thus exists a tradeoff between these two criteria, however, a two inch separation distance for adults and a one inch separation distance for children is considered a valid compromise. As noted, the separation distance between the long sides 51, 53 of the rectangle will not affect or change the frequency content of biopotential signals A and B, as long as the source of the electric current lies in the same orientation to both axis lines. Only an attenuation in amplitude between EKG measurements of A and B would be detected.

The difference between the parallel vectors is represented by the C-D difference relationship. The equation is reformatted as follows: ∥B∥U_{B}−∥A∥U_{A}=C-D. Since U_{A} and U_{B} are parallel and linear to each other we can combine them into a single variable, thus eliminating a variable from the equation. Therefore, (∥B∥−∥A∥) U=C-D. The factor (∥B∥−∥A∥) is a scalar representing the difference in length between the two vectors. The attenuation factor x=∥A∥/∥B∥. This scale factor, x, can be measured during a one-time single calibration step when first placing the sensors on the body through use of a hardwire connection between first and second sensors, and its value retained in memory for future use. Initial hardwire calibration of the scale factor is only needed for optimum results to recreate the calculated signals with maximum fit to a traditionally measured signal. Recalibration is only needed for optimum results when replacing the physical electrode patches on the body with new ones in order to correct for any misplacement errors compared to the exact previous location of the contacts. Therefore, to find B, where is the desired lead biopotential measurement along the axis line of the lead of interest, and remembering B=∥B∥U_{B}, a calculation is performed as follows:

(∥B∥−x∥B∥) u=(C-D)

∥B∥u=(C-D)/(l−x) or

B=(C-D)/(l−x) where C, D, and x are all known (measured) variables.

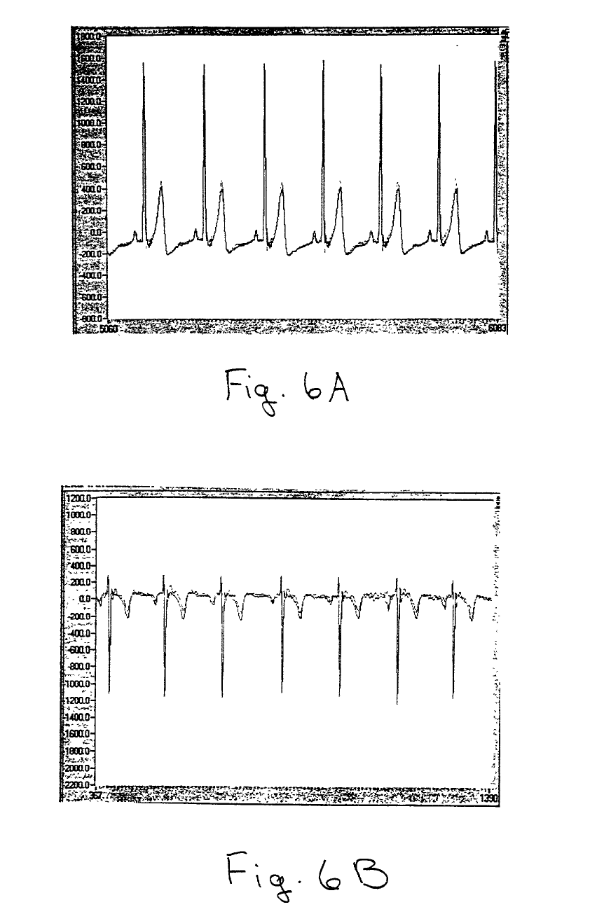

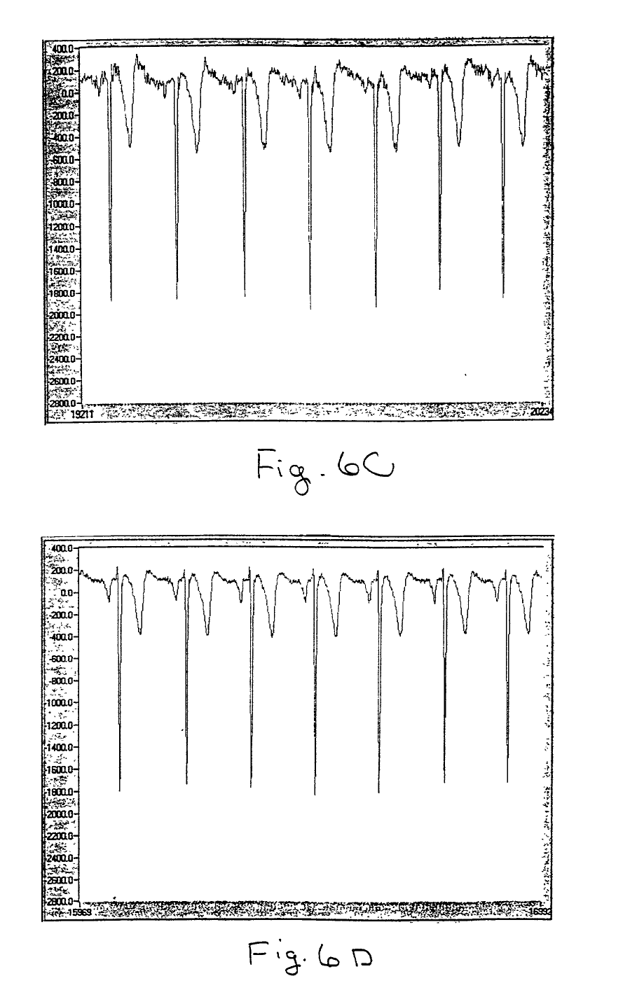

B is a calculation which is a close approximation, or equivalent to, the standard EKG lead measurement along the axis line of the long side of the rectangle 49. For lead 1, this axis line connects LA with RA locations on patient body. A and B are thus aligned horizontally, and C and D are aligned vertically. FIG. 6A depicts the computed versus measured values of Lead I.

The same process is applied to measure Lead III (FIG. 3B), except the long side of the rectangle lies along the axis line connecting LA, with the LL locations on the patient body. A and B are thus aligned vertically, and C and D are aligned horizontally. FIG. 6B depicts the computed versus measured values of Lead III.

From the closed circuit loop comprised of Lead I, Lead III, and Lead II, we can then calculate Lead II from the measured Lead III, and Lead I, (Lead II=Lead I+Lead III). FIGS. 6C and 6D depict computed and measured values of Lead II, respectively.

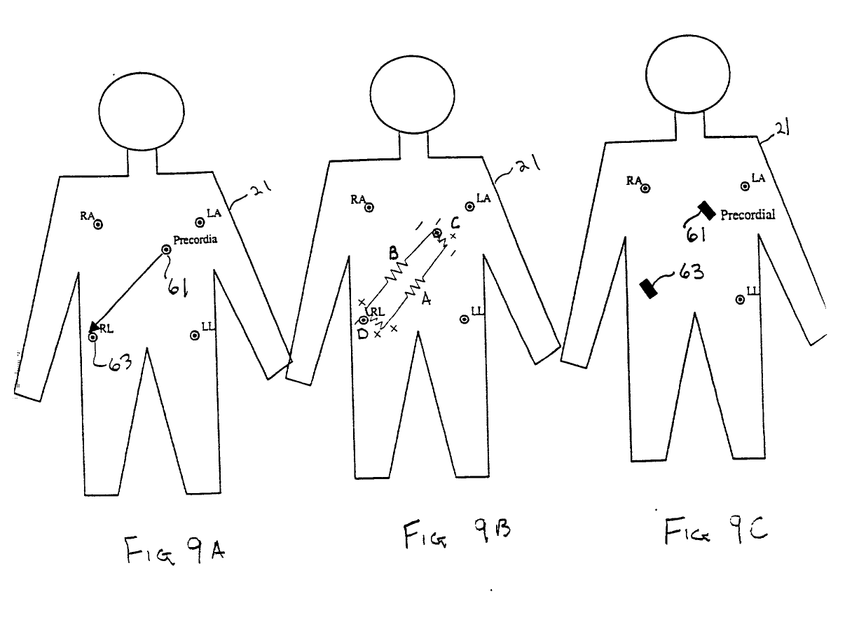

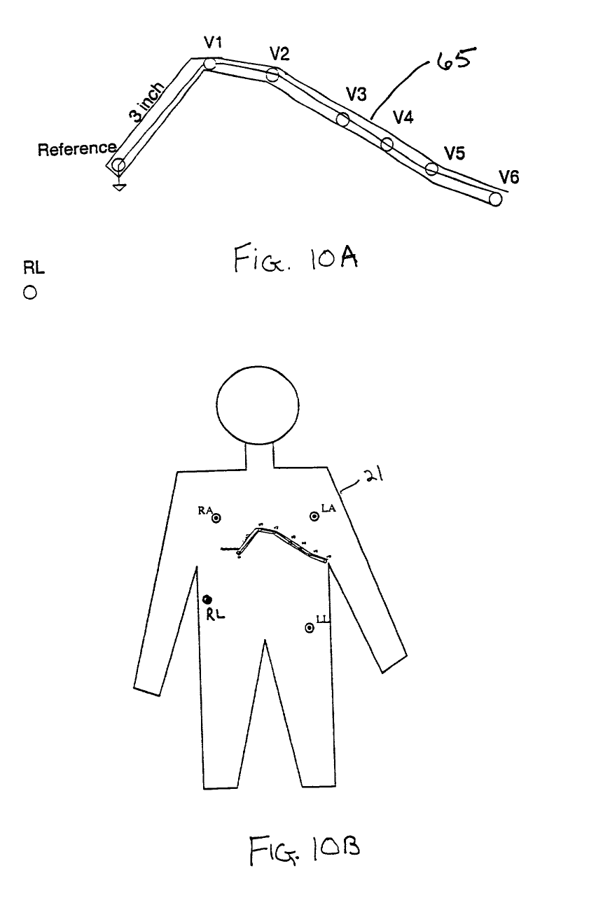

Referencing FIGS. 9A-9C, the same concept can be applied for obtaining an additional precordial lead in a 4-lead EKG system by applying a linear line electrode 61 on top of the heart and another line electrode patch 63 to the right leg area RL to produce a precordial measurement. In a standard 12-lead EKG, leads aVL, aVR, and aVF (not shown) are computed from measurements based on Leads I, II, and III represent augmented left arm, augmented right arm, and augmented left foot measurements between the LA, RA, LL areas and the average value of Leads I, II, and III. These three computed leads can be provided by the present invention along with standard 3-lead measurement systems as they require no additional measurement instrumentation.

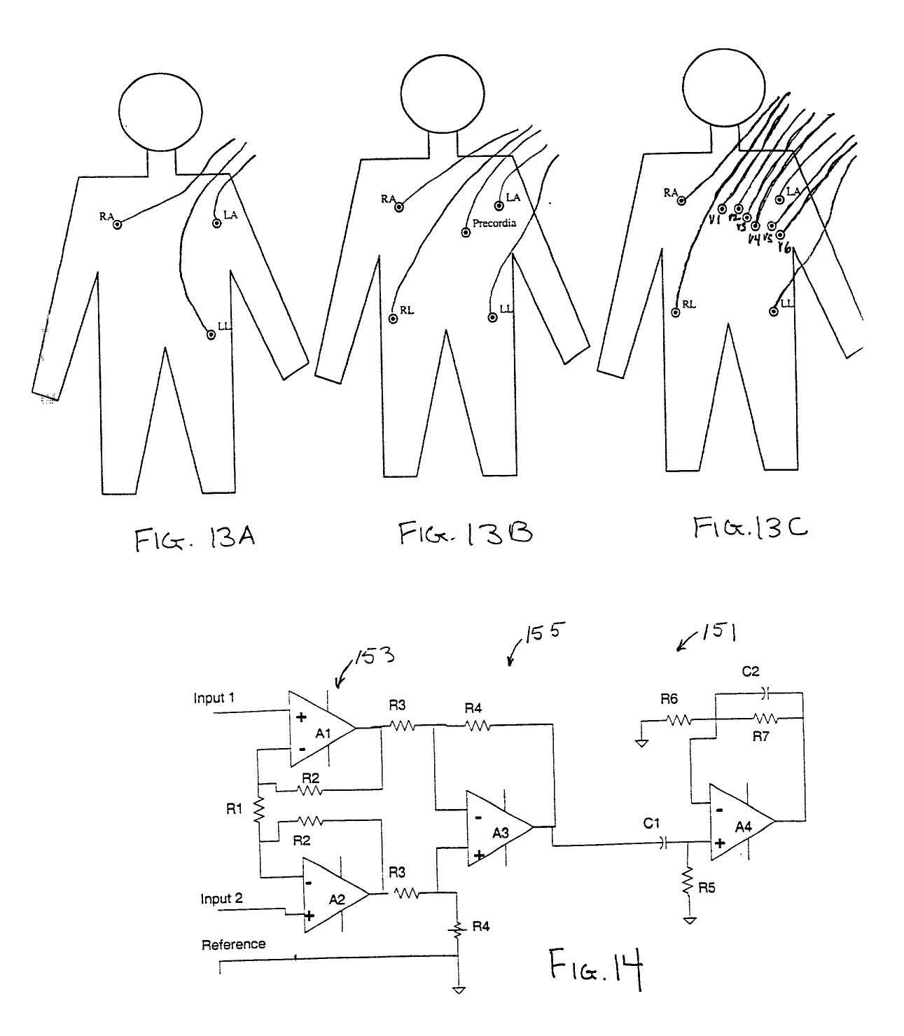

Referencing FIG. 11, for a 12-lead EKG system, one must measure six precordial leads V1, V2, V3, V4, V5, V6. The present invention uses a strip of electrodes 65 that measures the potential between each of the electrodes V1-V6 and a common reference point 67 located on the right half of the body, along the axis of direction of RL, as indicated, and within about three inches the of location of V1. The described separation provides an excellent reproduction of the precordial leads without having to extend the reference point all the way to the RL site on the right hip area. By contrast, FIGS. 13A-13C show a prior art conventional EKG measurement technique for 3-lead, 4-lead, and 12-lead EKG with electrical wires attached.

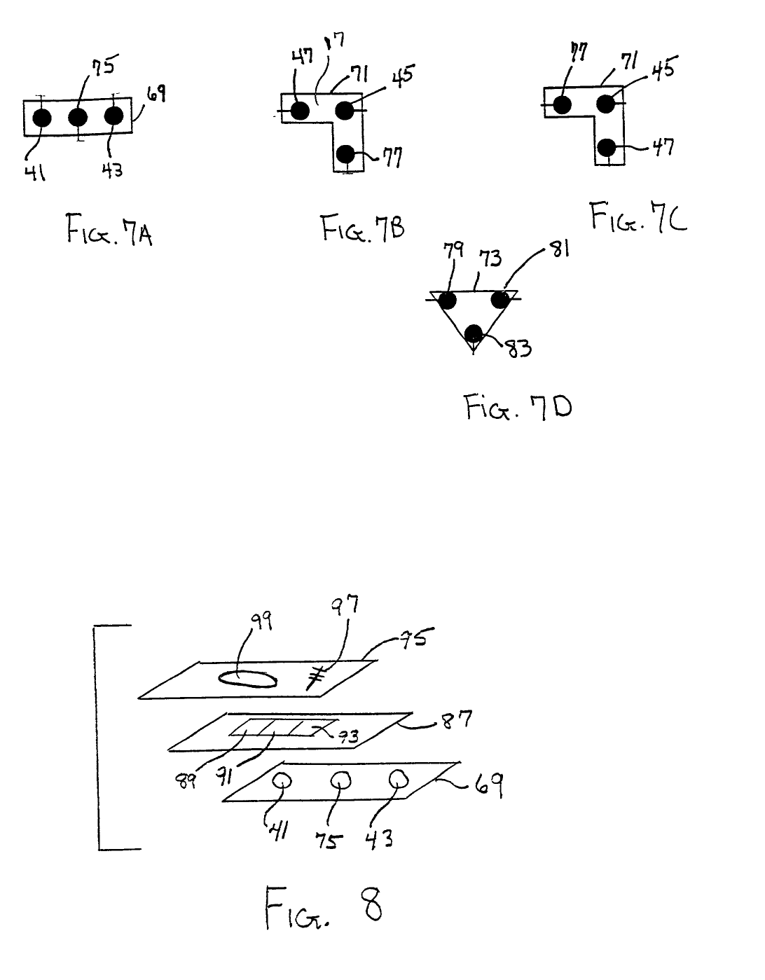

FIGS. 7A-7D show three preferred configurations 69, 71, 73 for the three-point contact electrode patches. One preferred configuration is a linear straight line patch 69 (FIG. 7A) with the two outermost contacts 41, 43 representing the two inputs and the middle contact 75 used as a common local reference point. Another preferred configuration is a right angle configuration 71 where one side, or arm, 17 of the patch containing two contacts is used for the two inputs 45, 47 and the third contact point 77 is used as a common local reference. This configuration 71 can be useful at the left arm position, for example, using the side two contacts 45, 47 vertically (FIG. 7C) for measurement of lead I, and alternatively using the side contacts horizontally (FIG. 7B) for measurement of lead III. A third preferred embodiment is a three point contact electrode 73 where the three electrode points are arranged in an equilateral triangle shape with two inputs 79, 81 and a common local reference electrode 83. The contact points on the electrode patches will have snap-on conductive metal sites for attachment directly to the sensors. Such contacts are preferably made from silver or are silver coated.

Referencing FIG. 8, there are depicted multiple layers suggested as comprising the fabrication of the sensor portion of the wireless EKG system. First the electrode adhesive layer patch, e.g. in-line patch 69, attachable to the patient skin, contains three conductive contact points 41, 43, 47 and silver-chloride electrolyte on an adhesive web 85 such as a Mylar ™ web coated with a suitable known adhesive. The electronic circuit sensor assembly layer 87 contains a miniaturized or integrated circuit, or both, show as sections 89, 91, and 93, on top of the electrode patch. The circuit layer 87 snaps into position and electrical contact by the snap-on contact conductive metallic points on the electrode layer as mentioned above. The top layer 95 is a cap, or cover, which protects the electronic assembly and acts as an antenna 97 or has an antenna embedded within it. The cap, or cover also contains a battery 99 that is preferably replaceable when needed, as indicated by a base station indicator for sensor battery status or by a light emitting source on the sensor itself.

FIG. 11 depicts a block diagram of the electronic assembly components of the sensors. The sensor assembly 101 is composed of preamplifier channels 103, 105 for each of the two input signals measured with respect to the common reference signal 107. The preamplified outputs 109, 111 from the two channels 103, 105 are passed to a differential instrumentation amplifier 113 which amplifies the differential of the two input signals into an output signal 115 that has much of the common noise components eliminated. The amplified output 115 is then passed to an antialiasing filter 117 which band limits the measured output signal to within a frequency band that is at most half of the sampling frequency of an A/D converter 119 which receives the antialiasing filter output. A 60 Hz notch filter (not shown) can be also added to minimize 60 Hz power line interference. The A/D converter 119 then samples and digitizes the signal, and its output 120 passes the digitized values to the sensor microcontroller 121. The sensor microcontroller 121 stores the digital signal values in random access memory 123, and further applies filtering and enhancing signal processing and conditioning using a digital signal processor (DSP) processor 125 and also passes the resultant conditioned signals to the wireless, e.g. RF, transceiver 127. The sensor microcontroller 121 then modulates and encodes the digital values prior to transmitting them through the antenna 97. Each sensor can also receive incoming command messages from the base station via the antenna 97 and demodulate and decode the incoming wireless transmissions. Such incoming decoded messages are then processed and analyzed by the microcontroller 121 in order to interpret commands and decide on and execute corresponding action. The sensor may also respond to such commands or informational messages from the base station 129 by building and transmitting a corresponding response message, if needed, in accordance with a predefined communication protocol that defines the exchange of messages and information between the sensors and the base station. Each of the sensors is powered by the DC battery source 99.

FIG. 14 depicts a conventional instrumentation amplifier circuit 157 that is preferably incorporated into the electronic assembly of each sensor, e.g. 11. This instrumentation amplifier 151 offers a high common mode rejection ratio and multiphase amplification, with high input impedance. Input 1 and Input 2 are bipolar input channels, and the reference is a common potential. The amplifier has high input impedance, and high common mode rejection ratio CMRR. The differential gain is determined by the resistors in the two amplifier stages 153, 155. The first stage 153 has two non-inverting amplifiers A1, A2 that are coupled by a common resistor R1 to provide very high input impedance. The second stage 155 is conventional differential amplifier A3. Two stage differential amplifiers will enhance performance in removing common signals such as 60 Hz, and measure differential value between the two contact inputs. Resistor R4, across one input and the output of the differential amplifier A3, can be adjusted for maximizing CMRR. This design results in desired amplification gain distributed over multiple stages of the amplifier. A third stage amplifier A4 receives the output of differential amplifier A3 and acts as a filter to limit the bandwidth of the measured EKG waveform.

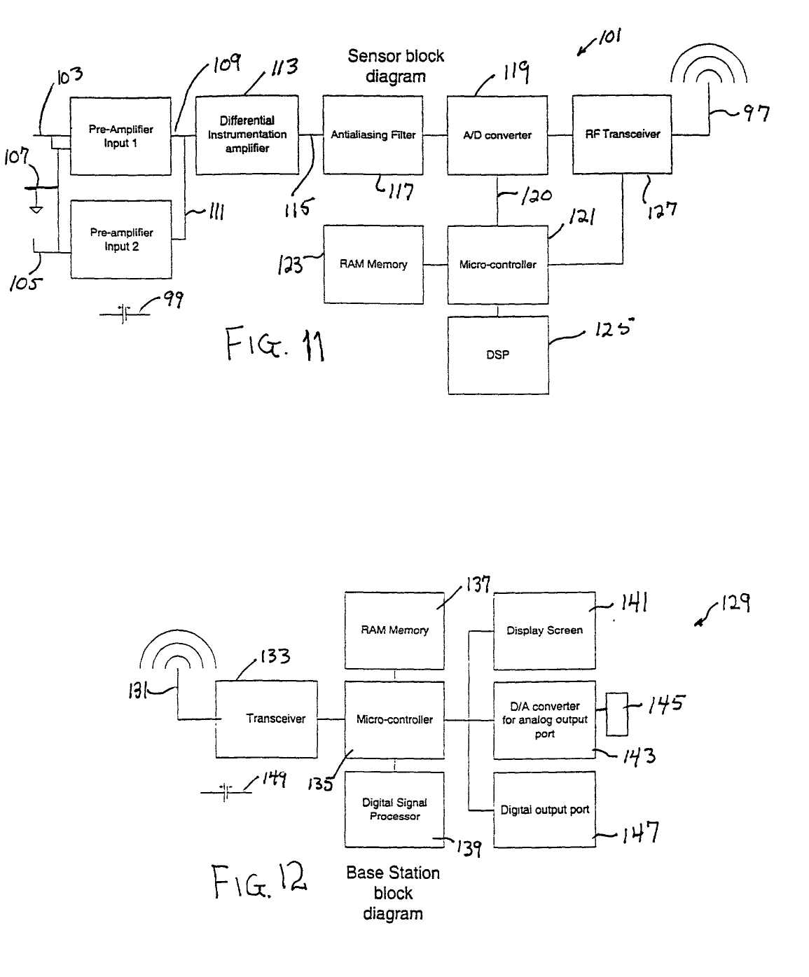

Referencing FIG. 12, the base station electronic assembly 129 block diagram comprises an antenna 131 that receives incoming wireless transmissions of data or response messages from the sensors, e.g. 11, 13, 29 (FIG. 1A) and passes them to its wireless transceiver 133. The transceiver 133 decodes and demodulates incoming wireless transmissions and passes the decoded data to the base station's microcontroller 135 which analyzes and interprets the incoming messages and decides and executes response logic, if needed. The microcontroller 135 may then store incoming informational messages and processing data into RAM memory 137 and/or perform further signal processing and conditioning on incoming data blocks using the DSP 139. The base station microcontroller 135 can also display the incoming data on a local display monitor screen 141. The incoming data can also be converted from digital format into analog via a D/A converter 143 and output at an analog output port 145 in order for the data to be transferred or interfaced to standard monitors in the hospital for example. Alternatively, the data can be output to a digital output port 147 in order to interface to a personal computer (PC) (not shown) which would then display the incoming EKG signals using PC software. The base station microcontroller 135 can also build informational messages and commands and send them to the wireless transceiver 133 where they are encoded, modulated and transmitted to the sensors using the antenna 131. The base station is preferably powered by a dual power supply source using either the battery or an external DC power supply source 149.

The base station has a detachable adapter unit (not shown) that can connect on the base station analog output port 145 and which provides snap-on conductive sites for attachment of electrodes from standard EKG monitoring equipment in order to simulate a standard EKG source for the monitoring equipment. This adapter can be of different designs to provide a 3-lead, 4-lead, or 12-lead EKG analog output channels if desired.

The base station is configured to display individual EKG leads from current (realtime) or previously stored data as a historical data review feature. It is also configured to display the patient's pulse rate, and detect and indicate any abnormal anomalies in the patients EKG. When continuously monitoring patients for an extended period of time, the base station may analyze, identify, and locate the abnormal events in a patient EKG in order to filter down the amount of data that needs to be reviewed by the physician. The base station may also store patient demographics, emergency contact information and physician information in its memory. The base station can also be enabled with an internet connection via a wired modem port, or a wireless internet connection port which allows for the EKG data to be formatted and transferred directly on-line to a physician, and allows for sending notification in case of emergency to a patient's contacts, physician, and local authorities. The base station can be further equipped with a commercially available touch screen for ease of user interface when selecting user choices from the options provided by the software on the base station. The touch screen can be further lit with background light for operation during the night if desired.

Multiple base stations can also communicate two way with each other in order to exchange all patient-specific information such as patient, physician, sensor, and sensor group identification information, as well as calibration data and configuration information to allow transparent transfer of patient EKG data from one base station to another without having to disconnect the sensors from the patient. Each base station may sense signals available from all available sensors in the region of communication and may display patient identifying information on the display. Once a patient is selected, then the transfer process is initiated, and then control and EKG data transfer is completed to the new base station, and the previous base station stops its control over the sensors.

The base station is also able to integrate newly added wireless sensor data and function identifying information into its group of existing sensors on-the-fly by wireless over-the-air programming, whereby the new wireless sensor can provide additional physiologic data of interest such as from sensors for measuring an additional EKG lead, EMG lead, EEG lead, EOG lead, or vital signs such as body temperature, respiration rate, blood pressure, blood oximetery, and tidal CO₂, and blood sugar levels or other biochemical and biophysical parameters.

In general, due to the fact that the separation between the contact points on the electrodes is much smaller than the conventional electrode measurement, the measured signals are of much smaller biopotential amplitudes, and therefore require that the signals be amplified at a higher gain than conventional EKG systems. Also since the system is taking the difference between two measurements, the measurements have to be sampled simultaneously so that the subtraction process results in accurate results. Such synchronized simultaneous timing for sampling is administered and controlled by the base station via a timing program. The base station also synchronizes the data transmission timing across the multiple sensors by time division multiple access. Each sensor transmits its data in its dedicated time slot. The time base between the base station and the individual sensors is synchronized by using either a time base signal from the base unit to the sensors or by sharing common precise timing source crystals from which timing can be synchronized. The wireless communication between the sensors and the base station is either a time division multiple access (TDMA) technology where the transmission occurs on a single carrier frequency channel where the sensors communicate only on an specific timeslot on that frequency, or in a preferred embodiment, a code division multiple access (CDMA) communication technique where the communicated information is spread over a wide band of frequency range as encoded by an identifying code. The two schemes, TDMA and CDMA, are conventional to modern communication devices and easily adapted to the present invention.

The base unit preferably has two interface types, i.e. one or more digital I/O ports, and one or more analog output ports, to interface to personal computers or standard EKG monitors, for display and analysis of the data. The EKG monitor can be a conventional, standard monitor typically used in a hospital setting. A detachable adapter can be connected to the base station, to provide analog output interface connection sites for snapon leads from conventional monitors to connect to. The signal provided at the output is in analog form, and thus undergoes a D/A conversion at the base station after being received in digital format from the sensors. The signals will therefore have to be converted from analog to digital and back to analog at the base station, and then may go through yet another analog to digital conversion at the conventional monitor itself, resulting in loss of resolution. The data acquisition sampling rate of the present invention is therefore much higher at the sensor than current conventional monitor sampling rates, in order to provide a high quality analog output signal (smooth, not square wave shaped) of standard format from the base station to the interfacing monitors after two phases of quantization and sampling. Therefore both the bit resolution for A/D (preferably 16 bit or more) and the sampling rate (preferably 5000 kHz or higher) has to be higher at the sensor.

The individual wireless sensors are each pre-programmed with a unique sensor identifier. In a preferred embodiment, the multiple individual wireless sensors are pre programmed with a specific functional location on the patient body such as left arm, right arm, left leg, etc. which would eliminate possibility of error on the user part during placement, as well as eliminate additional overhead time consumed during configuration. The sensors can be however reprogrammed over the air with a specific functional position assignment. Also, the sensor can be programmed over-the-air with the associated base station's unique identifier, the group identifier of the associated group of sensors on one patient, and the patient identification information.

The monitoring system operation is described as follows: first, the adhesive three point contact electrode patches are attached to the patient body on the proper anatomical location, and in the proper positioning orientation. The sensors are then snapped-on a detachable adapter of the base station analog output interface in order to provide a direct electrical connection with the base station. The base station then places a high volt DC signal on the output analog port to all the sensor channels to start the initialization procedure. The sensors can then accept programming with new configuration information via informational messages received by wireless interface. Once configured, the sensor identification and functional position information can be stored in the base station's memory. Similarly, the base station identification, sensor group identification, patient information, physician information, and emergency contact information can all be stored in the sensor's memory. The base station will page the individual sensor transceivers, and once a response is received, a wireless connection is established. The sensors are then removed from the base station and connected to the selected patient at the previously placed electrode patch sites. If a response is not received from the sensors once on the patient, the base station will periodically page the sensors in an attempt to locate them and establish an wireless connection. If the wireless connection is lost, the sensor will remain dormant in operation and stop all data acquisition. When a page is received from the base station, the sensor sends a response indicating it is within communication range. The sensor will also send messages to the base station in response to commands or to transmit acquired EKG data continuously, as long as the wireless connection is established. A periodic auditing message between the sensor and the base station will indicate that the wireless connection is established.

Each of the sensors can be clearly labeled and colored for correct association with the anatomical location. The EKG technician can place the sensors on the patient body, and then instruct the base station to start receiving acquired EKG measurements from the identified group of sensors. The base station will request the sensors to initiate data acquisition and transmission. If the signal level is too low or noisy, the base station may request the sensors to adjust the amplification gain to obtain a better signal to noise ratio.

The sensor identification information, as well as the use of code division multiple access technology, will enable the system to uniquely distinguish between sensors attached to multiple patients in proximity. Signals received from sensor transceiver assemblies that are different from the associated and identified patient sensors will be rejected. Similarly, the sensors will be able to associate with and accept signals only from the base station that they are configured with.

In order to provide easy mobility of patient data from sensor groups between multiple base stations that can be attached with multiple monitoring stations, each base station will scan the air for potential sensor transmitters periodically, and once it detects a group of sensors, the patient identifying information will appear on the base station screen. This allows the technician to simply select the name of the patient on the new receiving base station, and then sensor association and control is transferred from the old base station to the new base station. The sensor configuration information and EKG data is thus routed to the new base station unit. A monitor connected to the new base station can then start receiving the EKG data.

Multiple repeater antenna units can be placed across a distance to boost the wireless signal power and strengthen the EKG communication channel signals, to extend the distance over which transmission between the sensors and the base station is possible. The EKG signals received at the base station can be stored in memory for many hours of recording, and can be later uploaded to a receiving data review station, storage media, or internet connection via the digital I/O port. A secured internet site connection can provide a way to transmit the stored or real-time EKG data from the patient directly to an internet site where it can be accessed for review or immediate on-line real-time monitoring of the patient by a physician.

When continuously monitoring patients with the described EKG system, a large amount of data is collected, and is presented for review. Multiple signal processing and analysis algorithms are run on the base station, examining acquired EKG signals for detection and identification of abnormalities or anomalies in the signals. The abnormal signal events are tagged for presence and type of abnormality. This will allow the physician to filter through data and to review more efficiently and productively the individual events obtained at specific time windows instead of the whole data set during an extended period of monitoring time. Such algorithms may include, but are not limited to, abnormal tachycardia, brady cardia, arrhythmia, etc.

Novel apparatus and methods to acquire biopotential electrical signals have been described. Persons skilled in the art shall appreciate that the details of the preferred embodiment described above can be changed or modified without departure from the spirit and scope of the invention. The spirit and scope of the invention is to be limited only by the appended claims.

I claim:

1. A system of obtaining a calculated Lead I standard EKG measurement waveform from unconnected EKG sensors placed on a patient's body, comprising:
a) a base station comprising a wireless transceiver for two-way communication with multiple individual, wireless sensors;
b) Right Arm and Left Arm individual unconnected wireless sensors constructed and arranged for establishing a communication with the base station, each sensor adapted for receiving electrical contacts; the plurality of individual wireless sensors including:
ii. the Right Arm sensor having first and second input contacts defining a first line, with a voltage drop C, between the first sensor first and second input contacts; and
ii. the Left Arm sensor having first and second input contacts defining a second line, with a voltage drop, D, between the second sensor first and second input contacts;
c) whereby the Right Arm sensor first and second input contacts and the Left Arm sensor first and second input contacts define a quadrilateral when placed on a patient's body with the first and second lines being two sides of the quadrilateral;
d) a third line and a fourth line of the quadrilateral being parallel;
e) the third line of the quadrilateral extending between the first sensor first contact and second sensor first contact and having an EKG voltage drop, A;
f) the fourth line of the quadrilateral extending between the first sensor second contact and second sensor second contact having an EKG voltage drop, B;
g) the sensors adapted to obtain and send a measurement of the C and D voltage drops to the base station, and
h) the base station adapted to receive the measurement of the C and D voltage drops from the sensors and calculate the EKG voltage drop B, representing Lead I, by using the measured C and D voltage drops and an attenuation value x, between the amplitude of A and the amplitude of B.

2. The system of claim 1 wherein the EKG voltage drop B is calculated by performing the calculation:
B=(C-D)/(1−x), where (1−x) is a calculated scaling factor.

3. The system of claim 2 wherein x=∥A∥/∥B∥, with ∥A∥ being the norm value representing the length of a vector for A and ∥B∥ being the norm value representing the length of the vector for B.

4. The system of claim 1 wherein the attenuation value x is obtained by taking conventional wired EKG voltage drop measurements of A and B, via a temporary hard wire connection and calculating the attenuation between the A and B EKG voltage drops to determine the attenuation value x, storing the attenuation value x in a memory of the base station and removing the hard wire connection.

5. The system of claim 1 further including a Left Leg unconnected EKG sensor adapted to be placed on the body in relation to the Right Arm and Left Arm EKG sensors for obtaining a Lead III EKG waveform,
wherein the Lead III EKG waveform is calculated from a first measurement of differential potential value between two input contacts of the Left Arm sensor and a second measurement of differential potential value between the two input contacts of the Left Leg sensor;
the second measurement being subtracted from the first measurement and the result of the subtraction being divided by a scaling factor, where the scaling factor includes an attenuation ratio value between two previously measured standard EKG measurement waveforms taken between the Left Arm and Left Leg sensors.

6. The system according to claim 5 wherein the Left Leg sensor has first and second input contacts and a third reference contact.

7. The system according to claim 5 wherein the two previously measured standard EKG measurement waveforms taken between the Left Arm and Left Leg sensors are a first measurement taken between the first input contacts of each of the Left Arm and Left Leg sensors, and a second measurement between the second input contacts of each of the Left Arm and Left Leg sensors.

8. The system of claim 5 further including:
the base station being adapted to calculate a Lead II EKG waveform value between the Right Arm sensor and the Left Leg sensor, wherein the Lead II EKG waveform value is calculated from the Lead I EKG waveform and the Lead III calculated EKG waveform as indicated by the following relationship: Lead II= Lead I+ Lead III.

9. The system of claim 1 wherein the electrical contacts include two input contacts and a reference contact.

10. A system of obtaining a calculated standard biopotential measurement waveform from unconnected biopotential sensors adapted to be placed on a patient's body, comprising:
a) a plurality of individual unconnected wireless sensors constructed and arranged for establishing wireless communication, each sensor having first and second electrical contacts; the plurality of individual sensors including:
i. a first sensor having its first and second contacts defining a first line, with a voltage drop C, between the first sensor first and second contacts; and
ii. a second sensor having its first and second contacts defining a second line, with a voltage drop, D, between the second sensor first and second contacts;
b) whereby the first sensor first and second contacts and the second sensor first and second contacts define a quadrilateral when placed on a patient's body with the first and second lines being two sides of the quadrilateral;
c) a third line and a fourth line of the quadrilateral being parallel;
d) the third line of the quadrilateral extending between the first sensor first contact and second sensor first contact and having a voltage drop, A;
e) the fourth line of the quadrilateral extending between the first sensor second contact and second sensor second contact having a voltage drop, B;
f) the sensors adapted to obtain a measurement of the C and D voltage drops;
g) each sensor adapted to communicate its respective C or D value to a data processing unit; and
h) the data processing unit adapted to receive the measurement of the C and D voltage drops from the sensors and calculate the voltage drop B, representing the desired standard biopotential measurement voltage drop, by using the measured C and D voltage drops and an attenuation value x.

11. The system of claim 10 wherein the voltage drop B is calculated by performing the calculation:
B=(C-D)/(1−x).

12. The system of claim 11 wherein x=∥A∥/∥B∥, with ∥A∥ being the norm value representing the length of a vector for A and ∥B∥ being the norm value representing the length of the vector for B.

13. The system of claim 11 wherein (1−x) is a calculated scaling factor between the amplitude of A and the amplitude of B.

14. The system of claim 13 further comprising a memory adapted for storing the scaling factor, the memory being in communication with the data processing unit.

15. The system of claim 10 wherein the attenuation value x is obtained by taking conventional wired biopotential signal measurements, via a temporary hard wire connection between the contacts of the third line, and a temporary hard wire connection between the contacts of the fourth line, and calculating the attenuation between the A and B voltage drops to determine the attenuation value x and removing the hard wire connection.

16. The system of claim 10 wherein the data acquisition of the sensors is simultaneous.

17. The system of claim 10 wherein the electrical contacts for at least one sensor include two input contacts and a reference contact.

18. A system of obtaining a calculated standard biopotential measurement waveform with first and second unconnected biopotential sensors placed on a patient's body, wherein:
the standard biopotential measurement is calculated using a first measurement of differential potential value between two inputs of the first sensor and a second measurement of differential potential value between two inputs of the second sensor; the second measurement being subtracted from the first measurement and the result of the subtraction being divided by a scaling factor, where the scaling factor includes an attenuation ratio between two previously measured standard biopotential measurement waveforms.

19. The system of claim 18 wherein the scaling factor is 1 minus the attenuation ratio.

20. The system of claim 18 wherein each sensor has three electrical contacts, the three contacts including two input contacts and a reference contact.

21. A system of obtaining a calculated standard biopotential measurement waveform from unconnected biopotential sensors placed on a patient's body, comprising:
a) a base station comprising a wireless transceiver for two-way communication with multiple individual, wireless sensors;
b) a plurality of individual unconnected wireless sensors constructed and arranged for establishing a communication with the base station, each sensor adapted for receiving three electrical contacts including first and second input contacts and a reference contact; the plurality of individual wireless sensors including:
i. a first sensor having its first and second input contacts defining a first line, with a biopotential signal C, between the first sensor first and second input contacts; and
ii. a second sensor having its first and second input contacts defining a second line, with a biopotential signal, D, between the second sensor first and second input contacts;
c) whereby the first sensor first and second input contacts and the second sensor first and second input contacts define a quadrilateral when placed on a patient's body with the first and second lines being two sides of the quadrilateral;
d) the third line and the fourth line of the quadrilateral being parallel;
e) the third line of the quadrilateral extending between the first sensor first contact and second sensor first contact and having a biopotential signal, A;
f) the fourth line of the quadrilateral extending between the first sensor second contact and second sensor second contact having a biopotential signal, B;
g) the sensors adapted to obtain and send a measurement of the C and D biopotential signals to the base station, and
h) the base station adapted to receive the measurement of the C and D biopotential signals from the sensors and calculate the biopotential signal B, representing the desired standard biopotential measurement waveform, by using the measured C and D biopotential signals and an attenuation value x, between the amplitude of A and the amplitude of B.

22. The system of claim 21 wherein the voltage drop B is calculated by performing the calculation:
B=(C-D)/(1−x).

23. The system of claim 21 wherein x=∥A∥/∥B∥, with ∥A∥ being the norm value representing the length of a vector for A and ∥B∥ being the norm value representing the length of the vector for B.

24. The system of claim 21 wherein the biopotential signal is a standard EKG Lead I signal, the first sensor is a Left Arm (LA) sensor and the second sensor is a Right Arm (RA) sensor.

25. The system of claim 21 wherein the attenuation value x is obtained by taking conventional wired biopotential signal measurements, via a temporary hard wire connection between the contacts of the third line, and a temporary hard wire connection between the contacts of the fourth line, and calculating the attenuation between the A and B biopotential signals to determine the attenuation value x, storing the attenuation value x in a memory of the base station and removing the hard wire connection.

26. The system of claim 21 further including a third unconnected biopotential sensor adapted to be placed on the body in relation to the first and second biopotential sensors for obtaining a second biopotential waveform,
wherein a second biopotential waveform is calculated from a first measurement of differential potential value between two inputs of the first sensor and a second measurement of differential potential value between two inputs of the third sensor;
the second measurement being subtracted from the first measurement and the result of the subtraction being divided by a scaling factor, where the scaling factor includes an attenuation ratio value between two previously measured standard biopotential measurement waveforms taken between the first and third sensors.

27. The system according to claim 26 wherein the third sensor has first and second input contacts and a third reference contact.

28. The system according to claim 26 wherein the two previously measured standard biopotential measurement waveforms taken between the first and third sensors are a first measurement taken between the first input contacts of each of the first and third sensors, and a second measurement between the second input contacts of each of the first and third sensors.

29. The system of claim 26 wherein the biopotential waveform is a standard EKG Lead III signal, the first sensor is a Left Ann (LA) sensor and the second sensor is a Left Leg (LL) sensor.

30. The system of claim 26 further including:
the base station being adapted to calculate a third biopotential waveform value between the second sensor and the third sensor, wherein the third biopotential waveform value is calculated from the first calculated biopotential waveform and the second calculated biopotential waveform as indicated by the following relationship: third waveform value= first waveform value+ second waveform value.

31. The system of claim 30 wherein the third biopotential waveform is a standard EKG Lead II signal.
